(19) United States
(12) Patent Application Publication
(10) Pub. No.: US 2009/0082575 A1
(43) Pub. Date: Mar. 26, 2009

(54) PROCESS FOR PRODUCTION OF 2-CHLORO-4-NITROIMIDAZOLE

(75) Inventor: Koichi Shinhama, Tokushima (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: OTSUKA PHARMACEUTICAL CO., LTD., Tokyo (JP)

(21) Appl. No.: 11/663,724

(22) PCT Filed: Sep. 27, 2005
(86) PCT No.: PCT/JP2005/018230

§ 371 Date: Jul. 24, 2008

(30) Foreign Application Priority Data
Sep. 27, 2004 (JP) 2004-278974

(51) Int. Cl. C07D 233/93

(52) U.S. Cl. 548/327.1

(57) ABSTRACT

The present invention provides a process for production of 2-chloro-4-nitroimidazole in a high yield and at a high purity by a simple operation in a safer manner involving a low risk of explosion or the like. The present invention provides a process for production of 2-chloro-4-nitroimidazole represented by the formula (1): comprising a reaction of a 1-alkoxyalkyl-2-bromo-4-nitroimidazole compound represented by the general formula (7): wherein R1 represents a lower alkyl group, and n represents an integer of 1 to 3, with hydrogen chloride.

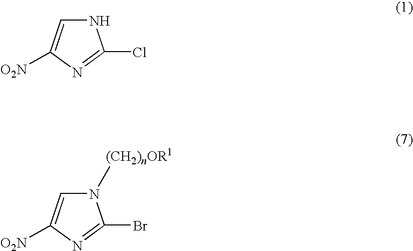

### TECHNICAL FIELD

The present invention relates to a process for production of 2-chloro-4-nitroimidazole.

### BACKGROUND ART

2-Chloro-4-nitroimidazole represented by the formula (I) is a compound useful as an intermediate for synthesis of various medicines, pesticides, etc., in particular, as an intermediate for production of an antituberculous agent.

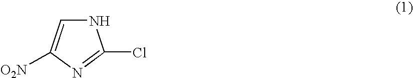

As a process for production of 2-chloro-4-nitroimidazole, processes shown in the following reaction formula-1 and reaction formula-2 have been conventionally known, for example (Jerzy SUWINSKI, Ewa SALWINSKA, Jan WATRAS and Maria WIDEL, Polish Journal of Chemistry, 56, 1261-1272 (1982)).

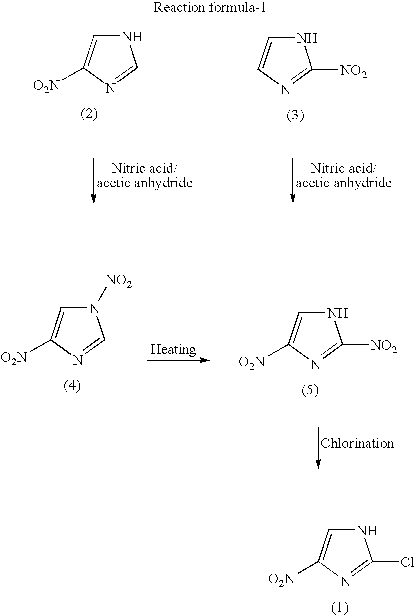

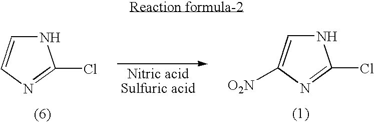

However, these processes have various drawbacks and are inappropriate as an industrial production process.

For example, in the process shown in the reaction formula-1, the compounds (4) and (5) as reaction intermediates are chemically unstable compounds, and are at risk of being exploded due to an impact by fall, friction, etc. Further, an industrial mass production of the target compound involves a high risk, because conversion of compound (4) into compound (5) by heating (at about 130° C.) is carried out at above TNR (Temperature of No Return: about 60 to 70° C., the maximum temperature which allows the compound to be handled with safety in an apparatus in a chemical process) of compound (4).

The process shown in the reaction formula-2 is a reaction of nitration of the compound (6). This nitration gives the compound (I) only in a low yield, and is industrially disadvantageous.

### DISCLOSURE OF INVENTION

An object of the present invention is to provide a process for production of high-yield and high-purity 2-chloro-4-nitroimidazole by a simple operation in a safer manner involving a low risk of explosion or the like.

As a result of conducting extensive studies for a safer and easier process for production of 2-chloro-4-nitroimidazole in order to achieve the above-described object, the present inventors have found that the object can be achieved by reacting a 1-alkoxyalkyl-2-bromo-4-nitroimidazole compound represented by the following general formula (7) with hydrogen chloride. The present invention has been accomplished based on such a finding.

The present invention provides a process for production of 2-chloro-4-nitroimidazole represented by the formula (I):

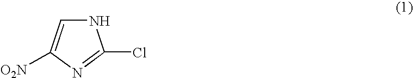

comprising a reaction of 1-alkoxyalkyl-2-bromo-4-nitroimidazole represented by the general formula (7):

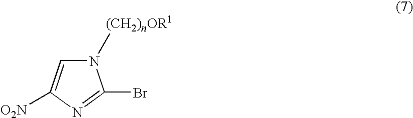

wherein R¹ represents a lower alkyl group, and n represents an integer of 1 to 3, with hydrogen chloride.

In the present invention, examples of the lower alkyl group include linear or branched alkyl groups having 1 to 6 carbon atoms such as methyl group, ethyl group, n-propyl group, isopropyl group, n-butyl group, isobutyl group, tert-butyl group, n-pentyl group, and n-hexyl group.

Process for Production of 2-chloro-4-nitroimidazole

The reaction of converting the compound represented by the general formula (7) into 2-chloro-4-nitroimidazole is carried out in an appropriate solvent or without a solvent in the presence of hydrogen chloride.

Although the amount of hydrogen chloride used in the above-described reaction is not specifically limited, hydrogen chloride is used typically in an amount of at least 2 moles, and preferably in a large excess amount per mol of the compound of the general formula (7).

Examples of the solvent used include water; lower alcohols such as methanol, ethanol, and isopropanol; ketones such as acetone and methyl ethyl ketone; ethers such as ethyl ether, dimethoxyethane, dioxane, tetrahydrofuran, and ethylene glycol dimethyl ether; fatty acids such as acetic acid and formic acid; esters such as methyl acetate and butyl acetate; N,N-dimethylacetamide, N-methylpyrrolidone, and a mixed solvent thereof.

The above-described reaction suitably proceeds typically at about 0 to 150° C., and preferably about room temperature to 100° C., and is generally completed in about 5 minutes to 40 hours.

The compound of the general formula (7) used as a starting compound in the present invention is produced by the following process, for example.

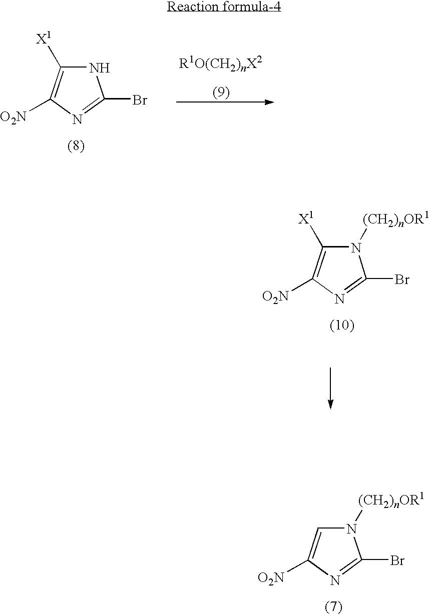

In the formula, R¹ and n are the same as above, X¹ represents a halogen atom, and X² represents a halogen atom or a lower alkoxy group.

Examples of the lower alkoxy group herein include linear or branched alkoxy groups having 1 to 6 carbon atoms such as methoxy group, ethoxy group, n-propoxy group, isopropoxy group, n-butoxy group, tert-butoxy group, n-pentyloxy group, and n-hexyloxy group.

The reaction of the compound (8) with the compound (9), wherein X² represents a halogen atom, is generally carried out in an appropriate solvent in the presence or absence of a basic compound.

Examples of the solvent used include aromatic hydrocarbons such as benzene, toluene, and xylene; ethers such as diethyl ether, dimethoxyethane, tetrahydrofuran, dioxane, and diethylene glycol dimethyl ether; halogenated hydrocarbons such as dichloromethane, dichloroethane, chloroform, and carbon tetrachloride; lower alcohols such as methanol, ethanol, isopropanol, butanol, and tert-butanol; acetic acid; esters such as ethyl acetate, methyl acetate, and butyl acetate; ketones such as acetone and methyl ethyl ketone; acetonitrile, pyridine, 2,4,6-collidine, dimethyl sulfoxide, N,N-dimethylacetamide, N,N-dimethylformamide, 1-methyl-2-pyrrolidinone (NMP), hexamethylphosphoric triamide, and a mixed solvent thereof.

Examples of the basic compound include inorganic bases including metal carbonates such as sodium carbonate, potassium carbonate, sodium bicarbonate, and potassium bicarbonate, metal hydroxides such as sodium hydroxide, potassium hydroxide, and calcium hydroxide, sodium hydride, potassium, sodium, sodium amide, and metal alcoholates such as sodium methylate and sodium ethylate; and organic bases including pyridine, 2,4,6-collidine, N-ethyldiisopropylamine, dimethylaminopyridine, triethylamine, 1,5-diazabicyclo[4.3.0]nonene-5 (DBN), 1,8-diazabicyclo[5.4.0]undecene-7 (DBU), and 1,4-diazabicyclo[2.2.2]octane (DABCO).

The basic compound is preferably used in an amount of typically 1 to 5 moles per mol of the compound (8).

The compound (9) is preferably used in an amount of typically at least about 1 mol, and preferably about 1 to 5 moles per mol of the compound (8).

The above-described reaction is carried out typically at about −50 to 200° C., and preferably at about −50 to 150° C. The reaction time is typically about 1 to 30 hours.

An alkali metal halide or the like such as sodium iodide may be added to the reaction system of this reaction.

The reaction of the compound (8) with the compound (9), wherein X² represents a lower alkoxy group, preferably employs acids including sulfonic acids such as camphorsulfonic acid, methansulfonic acid, and p-toluenesulfonic acid in place of the basic compound in the above-described reaction conditions. Of these, methansulfonic acid is preferable.

The acid is preferably used typically in a catalytic amount, and preferably in an amount of 0.01 to 0.2 mol per mol of the compound (8).

Further, P₂O₅ may be present in the reaction system.

The reaction of converting the compound (10) into the compound (7) is carried out in an appropriate solvent in the presence of a reducing agent.

Examples of the reducing agent used include metal sulfites such as sodium sulfite and sodium bisulfite; and hydride reducing agents including tetra-lower alkyl-ammonium borohydrides such as tetramethylammonium borohydride, tetraethylammonium borohydride, tetra-n-butylammonium borohydride, and tetra-n-butylammonium cyanoborohydride, sodium cyanoborohydride, lithium cyanoborohydride, sodium borohydride, and diborane.

Examples of the solvent used include water; lower alcohols such as methanol, ethanol, and isopropanol; ketones such as acetone and methyl ethyl ketone; ethers such as diethyl ether, dimethoxy ethane, tetrahydrofuran, diisopropyl ether, diglyme, and 1,4-dioxane; aromatic hydrocarbons such as benzene, toluene, and xylene; nitriles such as acetonitrile and propionitrile; dimethyl sulfoxide, N,N-dimethylformamide, N,N-dimethylacetamide, NMP, and a mixed solvent thereof.

When diborane or the like is used as the reducing agent, an anhydrous solvent is preferably used.

The reducing agent is preferably used in an amount of typically at least 1 mol, and preferably 1 to 10 moles per mol of the compound (10).

The above-described reaction is carried out typically at about 0 to 150° C., and preferably about 0 to 120° C., and is generally completed in about 1 to 30 hours.

The reaction of converting the compound (10) into the compound (7) may be carried out in an appropriate solvent in the presence of, for example, a catalytic hydrogen reducing agent such as palladium, palladium-black, palladium-carbon, palladium hydroxide-carbon, rhodium-alumina, platinum, platinum oxide, copper chromite, Raney nickel, or palladium acetate, and a fatty acid, fatty acid ammonium salt, or fatty acid alkali metal salt such as formic acid, sodium formate, ammonium formate, or sodium acetate.

As the solvent, any solvent used in a reaction using the above-described hydride reducing agent may be employed.

The catalytic hydrogen reducing agent is used in an amount of typically about 0.001 to 0.4 times, and preferably about 0.001 to 0.2 times of the compound (10) on a weight basis. The fatty acid, fatty acid ammonium salt, or fatty acid alkali metal salt is used in an amount of typically at least about 1 mol, and preferably about 1 to 20 moles per mol of the compound (10).

The reaction suitably proceeds typically at about room temperature to 200° C., and preferably about room temperature to 150° C., and is generally completed in about 1 to 30 hours.

An amine such as triethylamine, a phosphorus compound such as tri-o-tolylphosphine, or the like may be added to the reaction system.

The reaction of converting the compound (10) into the compound (7) may also be carried out in an appropriate solvent in the presence of a catalytic hydrogen reducing agent.

Examples of the catalytic hydrogen reducing agent include palladium, palladium acetate, palladium-black, palladium-carbon, palladium hydroxide-carbon, rhodium-alumina, platinum, platinum oxide, copper chromite, and Raney nickel. Such a catalytic hydrogen reducing agent is used in an amount of typically about 0.02 to 1 times of the compound (4) on a weight basis.

Examples of the solvent used include water; fatty acids such as acetic acid; alcohols such as methanol, ethanol, and isopropanol; aliphatic hydrocarbons such as n-hexane; alicyclic hydrocarbons such as cyclohexane; ethers such as 1,4-dioxane, dimethoxyethane, tetrahydrofuran, diethyl ether, monoglyme, and diglyme; esters such as methyl acetate, ethyl acetate, and butyl acetate; aprotic polar solvents such as N,N-dimethylformamide, N,N-dimethylacetamide, and NMP; and a mixed solvent thereof.

The reaction suitably proceeds typically at about −20 to 100° C., and preferably about 0 to 80° C., and is generally completed in about 0.5 to 20 hours. The hydrogen pressure is preferably about 1 to 10 atm, typically.

An amine such as triethylamine is preferably added to the reaction system. The above-described reaction advantageously proceeds by the addition of an amine.

The reaction of converting the compound (10) into the compound (7) may also be carried out in an appropriate solvent in the presence of a catalyst.

As the solvent, any solvent used in a reaction using the above-described hydride reducing agent may be employed.

Examples of the catalyst that can be used include palladium compounds such as palladium acetate-triphenylphosphine and tetrakis(triphenylphosphine)palladium. Such a catalyst is used in an amount of typically about 0.01 to 5 moles, and preferably about 0.01 to 1 mol per mol of the compound (10).

The reaction suitably proceeds typically at about room temperature to 200° C., and preferably about room temperature to 150° C., and is generally completed in about 1 to 10 hours.

An alkylsilane compound such as triethylsilane is preferably added to the reaction system. The above-described reaction advantageously proceeds by the addition of an alkylsilane compound.

In each of the above-described reduction reactions, selective dehalogenation occurs at the 5-position on the imidazole ring, so that the desired compound of the general formula (7) can be obtained.

The target compound obtained by the process of the present invention is easily isolated from a reaction mixture and purified by common isolation and purification means.

According to the present invention, high-yield and high-purity 2-chloro-4-nitroimidazole can be produced by a simple operation in a safer manner involving a low risk of explosion or the like.

### BEST MODE FOR CARRYING OUT THE INVENTION

The present invention will be explained in more detail below with reference to examples and reference examples.

### Reference Example 1

### Synthesis of 1-ethoxymethyl-2,5-dibromo-4-nitroimidazole

A mixture of 2,5-dibromo-4-nitroimidazole (20.0 g, 73.8 mmol), ethylal (100 ml), and methanesulfonic acid (1.42 g, 14.8 mmol) was stirred under heating (bath temperature: 65 to 70° C., internal temperature: 60° C., 1.5 hours). Further, the reaction mixture was evaporated under reduced pressure for two hours (fractional distillation column was used). The residue was allowed to cool to room temperature, and then ice water (200 g) was added, and the mixture was stirred for 10 minutes. The filtered crystals were washed with cold water and then air-dried (room temperature, 3 days). Thus, 1-ethoxymethyl-2,5-dibromo-4-nitroimidazole was produced.

Yield: 23.5 g (96.8%)

IR spectrum (KBr): 1532, 1491, 1464, 1397, 1365, 1344, 1315, 1273, 1248, 1127, 1106, 1054, 1020, 830, 740 cm⁻¹

¹H-NMR spectrum (CDCl₃) δ ppm: 1.25 (t, J=7.0 Hz, 3H), 3.64 (q, J=7.0 Hz, 2H), 5.50 (s, 2H).

### Reference Example 2

### Synthesis of 1-methoxymethyl-2,5-dibromo-4-nitroimidazole

A mixture of 2,5-dibromo-4-nitroimidazole (20.0 g, 73.8 mmol), methylal (100 ml), and methanesulfonic acid (1.42 g, 14.8 mmol) was stirred under water-cooling, and P₂O₅ (21.0 g, 148 mmol) was added to the mixture at below 42° C. Further, the mixture was suspended and refluxed under heating (43° C., 3 hours). The reaction mixture was evaporated under reduced pressure. The residue was allowed to cool to room temperature, and then ice water (200 g) was added, and the mixture was stirred for 10 minutes. The precipitated crystals were filtered, dispersed and washed (cold water 100 ml, 0.5 hour), and air-dried (room temperature, 3 days). Thus, 1-methoxymethyl-2,5-dibromo-4-nitroimidazole was produced.

Yield: 21.8 g (93.8%)

IR spectrum (KBr): 1543, 1530, 1486, 1458, 1439, 1367, 1318, 1260, 1194, 1119, 1104, 1053, 1013, 912, 833, 743 cm⁻¹

¹H-NMR spectrum (CDCl₃) δ ppm: 3.46 (s, 3H), 5.46 (s, 2H)

### Reference Example 3

### Synthesis of 1-methoxymethyl-2-bromo-4-nitroimidazole

1-Methoxymethyl-2,5-dibromo-4-nitroimidazole (12.5 g, 39.7 mmol) was dissolved in dimethylformamide (100 ml), and the solution was stirred under ice-cooling (12° C.). Further, water (50 ml) and sodium sulfite (10.0 g, 79.3 mmol) were added, and the mixture was stirred at room temperature (23 to 24° C.) for 72 hours. 5% Sodium bicarbonate aqueous solution (50 ml) and cold water (250 ml) were added, and the organic layer was extracted with ethyl acetate (250 ml, twice). The organic layer was washed with aqueous 5% sodium chloride solution (250 ml, twice), and then dried (MgSO₄) and evaporated (crystallization). Thus, 1-methoxymethyl-2-bromo-4-nitroimidazole was produced.

Yield: 8.17 g (87.2%)

Pale yellow crystals

HPLC 99.69%

IR spectrum (KBr): 3138, 1543, 1504, 1455, 1405, 1354, 1338, 1272, 1192, 1146, 1108, 1087, 1035, 989, 915, 824, 739, 668, 538 cm⁻¹

¹H-NMR spectrum (CDCl₃) δ ppm: 3.42 (s, 3H), 5.34 (s, 2H), 7.93 (s, 1H)

### Reference Example 4

### Synthesis of 1-ethoxymethyl-2-bromo-4-nitroimidazole

1-Ethoxymethyl-2,5-dibromo-4-nitroimidazole (13.1 g, 39.7 mmol) was dissolved in dimethylformamide (100 ml), and the solution was stirred under ice-cooling (12° C.) Further, water (50 ml) and sodium sulfite (10.0 g, 79.3 mmol) were added, and the mixture was stirred at room temperature (23 to 24° C.) for 72 hours. 5% sodium bicarbonate aqueous solution (50 ml) and cold water (250 ml) were added, and the organic layer was extracted with ethyl acetate (250 ml, twice; 100 ml, once). The organic layer was washed with a 5% sodium chloride aqueous solution (250 ml, twice), and then dried (MgSO₄) and evaporated. Thus, 1-ethoxymethyl-2-bromo-4-nitroimidazole was produced.

Yield: 8.74 g (88.0%)

Slightly yellow crystals

HPLC 98.51%

IR spectrum (KBr): 3139, 2983, 1540, 1507, 1455, 1400, 1340, 1279, 1264, 1163, 1138, 1096, 1038, 1009, 991, 828, 813, 741, 671 cm⁻¹

¹H-NMR spectrum (CDCl₃) δ ppm: 1.25 (t, J=7.0 Hz, 3H), 3.60 (q, J=7.0 Hz, 2H), 5.37 (s, 2H), 7.92 (s, 1H).

### Example 1

### Synthesis of 2-chloro-4-nitroimidazole

### One-Pot Process from N-Protected Compound

A mixture of 1-methoxymethyl-2-bromo-4-nitroimidazole (1.41 g, 5.96 mmol), concentrated hydrochloric acid (7.0 ml, concentration: 35%), and water (7.0 ml) was stirred under heating (at a bath temperature of 95 to 100° C. for 15 hours). The reaction mixture was evaporated under reduced pressure while maintaining the mixture at a temperature of 50° C. Water (8.4 ml) was added to the residue, and the mixture was stirred under cooling (at 5° C. for 1 hour). The crystals were filtered and dried by blowing air (at 60° C. for 15 hours) to obtain 0.641 g of the target 2-chloro-4-nitroimidazole (yield: 72.9%).

¹H-NMR spectrum (DMSO-d₆) δ ppm: 8.43 (s, 1H), 14.1 (br.s, 1H).

### Example 2

### Synthesis of 2-chloro-4-nitroimidazole

### One-Pot Process from N-Protected Compound

A mixture of 1-ethoxymethyl-2-bromo-4-nitroimidazole (4.05 g, 16.2 mmol), concentrated hydrochloric acid (20.3 ml, concentration: 35%), and water (20.3 ml) was stirred under heating (at a bath temperature of 97 to 102° C. for 12 hours). The reaction mixture was evaporated under reduced pressure while maintaining the mixture at a temperature of 70° C. Water (20 ml) was added to the residue, and the mixture was evaporated under reduced pressure. Further, water (20 ml) was added to the residue, and the mixture was stirred under cooling (at 5° C. for 1 hour). The precipitated crystals were filtered and then dried (at 60° C. for 16 hours) to obtain 1.41 g of the target 2-chloro-4-nitroimidazole (yield: 59.0%).

¹H-NMR spectrum (DMSO-d₆) δ ppm: 8.43 (s, 1H), 14.1 (br.s, 1H).

Further, the filtrate was concentrated to obtain 0.186 g of 2-chloro-4-nitroimidazole (yield: 7.8%).

Total yield: 66.8%

1. A process for production of 2-chloro-4-nitroimidazole represented by the formula: 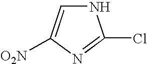
comprising a reaction of 1-alkoxyalkyl-2-bromo-4-nitroimidazole represented by the general formula: 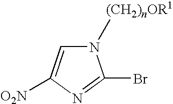
wherein R¹ represents a lower alkyl group, and n represents an integer of 1 to 3,
with hydrogen chloride.
